(19) United States
(12) Patent Application Publication
(10) Pub. No.: US 2010/0298627 A1
(43) Pub. Date: Nov. 25, 2010

(54) PHYSIOTHERAPEUTIC COMPLEX

(76) Inventor: Oleg Berezhkov, Moscow (RU)

Correspondence Address: Invest-C, LLC;c/o Oleg Berezhkov, P.O.Box 190897, Brooklyn, NY, 11219, US

(21) Appl. No.: 12/471,397

(22) Filed: May 24, 2009

(51) Int. Cl. A61M 21/00

(52) U.S. Cl. 600/27

(57) ABSTRACT

This invention is related to health and wellness equipment, and specifically can be used for medical and regenerative procedures assuming a complex influence of certain factors upon a patient psychological and physiological conditions under constant supervision of his condition. The physiotherapeutic complex containing a patient's seat with controllable height and back inclination angle, supplied by a control panel and built-in massage units, and also an audio-video system, the patient status control device, and a system control module. Innovation is that the complex is enhanced by a feeding air-oxygen-aroma mixture into the patient via a face mask or a nasal cannula.

### BACKGROUND

This invention can be used for medical, wellness, and regenerative procedures assuming a complex influence of certain factors upon a patient under constant supervision of his condition. The described complex allows to raise heart performance, promotes treatment of consequences of craniocerebral traumas, cerebral vascular diseases, borderline states of vegetodystonic type, diseases of nervous system, depressions. After analyzing the structure and operation functions of prior art it is necessary to note that known physiotherapeutic systems do not provide combination of controllable air-oxygen-aroma mixture, directly into the patient's, with other effecting modalities of controllable massage, audio-visual, and monitoring of a patient's psycho-physiological parameters. This invention solves the indicated problem and improves effectiveness of medical or preventive treatment because it is well-known that many malaises, fast fatigue, nervous breakdowns, heart instability and respiratory system damages are caused by oxygen deficit in organism. Using controllable level of oxygen gives synergistic influence upon the patient in combination with the other influence modalities. It underlines multifunctionality of the device, its convenience to the patient who takes an optimal position during the procedures. The patient's state is continuously monitored during all procedures.

### DESCRIPTION

Thus, the present invention aims at extending functionality and enhancing efficiency of psycho-physiological treatment of patients. The task is solved by the disclosed psycho-physiotherapeutic complex containing a patient's seat having adjustable height and back inclination angle, supplied by a control panel and massage units located in the patient seat, and also an audio-video system, the patient status control device, and a complex control module. Innovation is that the complex is equipped by an air duct system delivering air-oxygen mixture to the patient through a face mask or a nasal cannula. The complex can be equipped by an aromatizing device.

A rehabilitation psycho-physiological complex is equipped by audio tactile, music therapy and video-systems and an acoustic sensory armchair for the patient, and also a control panel containing block of gauges and electrodes connected to the patient, and a control unit. Sound frequency emitters are embedded in the armchair backrest and seat. They are situated aligned with the nape, backbone, and sciatic nerve zones. Emitters are made in the form of electrodynamic converters of electric fluctuations into sound, they are located in resonators cavities embedded in the armchair backrest and a seat of an armchair. Each converter has a diffuser with a juxtaposed sound channel connected to the armchair's cover which contacts the patient's body. The armchair backrest is hinged to the seat, allowing to change the backrest inclination angle or transfer it to horizontal position, forming a bed.

During therapeutic procedures the patient sits in the armchair, in a position most comfortable for him. The armchair backrest position is adjusted, electrodes and gauges connected to the patient body, and the complex systems is activated. Types of procedures and exposure modes are controlled from the control panel. It is natural that, depending on the chosen exposure type, all systems of the complex, or some of them, can be activated. Generally, the patient is exposed to sound waves through stereo-phones, he watches video images and undergoes massage. During the procedure, patient's state is constantly controlled.

### EXPLANATION OF OPERATION

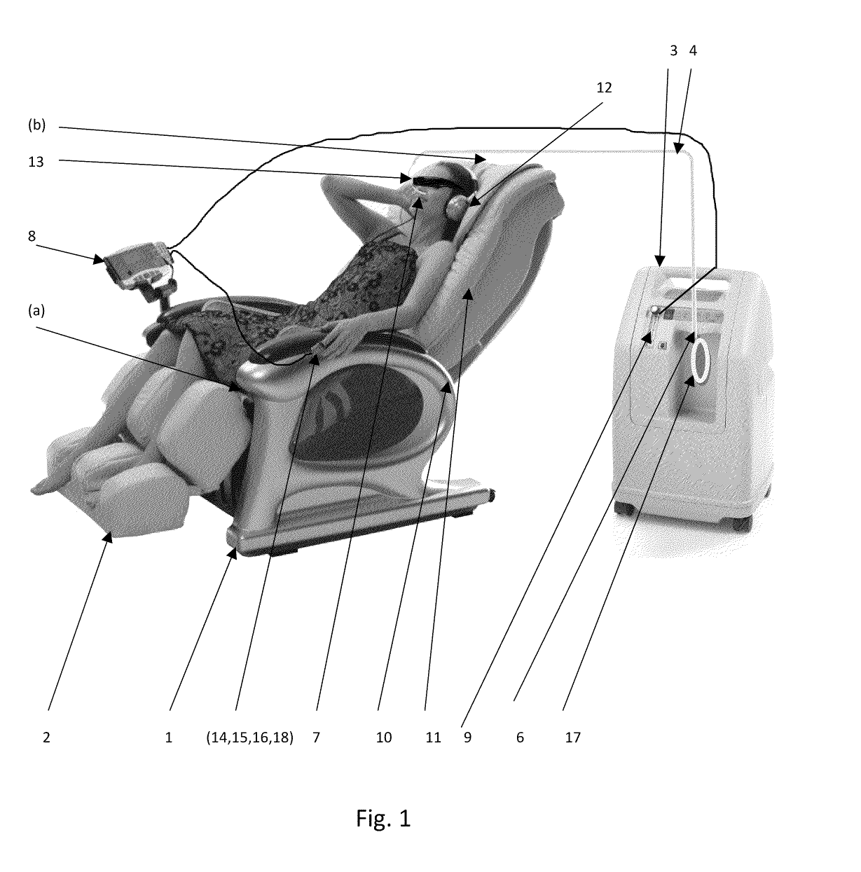

The essence of the invention is shown in FIG. 1. The physiotherapeutic complex consists of a seat 1 installed on a support with regulated height 2. The seat contains a seat (a) and a back (b). The complex is equipped by pipe 4 for supplying mixture of air and oxygen to a patient's organs of breath. One end of the pipe is connected to air mixer 6, and the other end of the pipe is connected to a respiratory mask or a nasal cannula 7. The mixer is a device supplying air to a respiratory mask or nasal cannula. It feeds air, oxygen or their mixture in some ratio. Construction of such mixers is available. The mixer 6 is controlled from control panel 8 connected to control unit 9. The control unit 9 is, as a rule, installed autonomously from the seat.

The back (b) of armchair 1 is turnable related to seat (a) by a hinge 10 for a preset angle, thus allowing the maximally comfortable position for the patient to be fixed. So the patient can totally relax which is necessary for some procedures. The back can take horizontal position related to the seat, so the seat can be transformed to a bed. Massage units 11 can be embedded into the back and/or seat equipped by drivers (not shown) allowing performance of active massage.

The complex is equipped by an audio system and sound columns 12 built in the seat back, and also a video system, the screen 13 located in front of the patient, its position is adjustable relatively to the patient's eye. The complex is equipped by various devices controlling the patient's condition: an electrocardiograph 14, pressure gauge 15 and pulse gauge 16. The complex can be equipped by an aroma emitter 17, and device 18 controlling the blood oxygen content. Operation of systems within the complex is controlled from control panel 8. Operating modes are set to the systems by control unit 9.

All systems and modules are composed of standard blocks. The physiotherapeutic complex functions as follows. We consider combinational operation of all its modules or selective operational functions can be used depending on a certain patient's state and prescribed treatment.

To carry out the procedure, the patient sits down in armchair 1. If necessary, armchair's height and inclination angle are adjusted. If necessary, an armchair is fold out the way to align the seat and the backrest to a single plane. All necessary electrodes and gauges are connected to the patient. Include complex systems. Procedures and their parameters (modes) are input in the control unit 9. Before carrying out the procedures, the patient's indices of heart rate, pulse and blood oxygen content can be measured. Procedure modes can be corrected if necessary, using those parameters.

The patient gets an oxygen mask or a nasal cannula 7. From the control panel 8 the amalgamator 6 is activated by control unit 9 that manages the process according to controllable parameters. When the breathing mixture is prepared in mixer 6, oxygen and air are mixed in a preset proportion to be inhaled by the patient through a nasal cannula 7. It is desirable to measure heart rate, pulse and blood oxygen content during the procedure. This data is transferred to the control unit. In a case of deviation of any parameter outside its control limits, the control unit switches the complex off.

As a rule, during oxygen therapy, air and oxygen are mixed in equal proportion (50% to 50% each component). Further during the procedure this ratio gradually changes to 70% of oxygen and 30% of air. This alteration is performed approximately within 15 minutes, then this mixture is fed for approximately 15 minutes, then during 15 minutes the ratio oxygen/air is 70%/30% is established.

In the process of therapy the patient is influenced by audio, receives video imagery, massage. If necessary, aromatizing is activated. This allows impart complex influence on respiratory, hearing, visual and smell organs in combination with a selective massaging of the patients body, whereby high therapeutic effectiveness of the treatment.

The complex can be used in restoration of functional capacities during post-operation and post-traumatic period, for restoration of workers involved in heavy physical work, athletes, for maintaining physical state of children, the handicapped and invalids suffering from limited movement lifestyle.

It will be apparent to those skilled in the art that various modifications and variations can be made to the structure of the present invention without departing from the scope or spirit of the invention. In view of the foregoing, it is intended that the specification to be considered as exemplary only, with a true scope and spirit of the invention being indicated by the following claims.

1. A physiotherapeutic complex, comprising:
a patient's seat having adjustable height and adjustable back inclination angle;
massage units located in the patient seat;
an audio-video system;
a control panel;
a patient psychophysiological parameters sensing module;
an air duct for feeding air-oxygen mixture into the patient's organism, said air duct is connected to an air-oxygen mixer on one end and to a face mask or nasal cannula on the other end.

2. The physiotherapeutic complex according to claim 1, having a built-in aroma therapy device allowing addition of controllable mix of aromatic substances into said air duct.
